(19) United States
(12) Patent Application Publication
(10) Pub. No.: US 2025/0368698 A1
(43) Pub. Date: Dec. 4, 2025

(54) THERAPEUTIC AND/OR PROPHYLACTIC ANTI-VIRAL AGENT

(71) Applicant: CENTRE FOR CELLULAR AND MOLECULAR PLATFORMS, Bengaluru, Karnataka (IN)

(72) Inventors: Paresh Brijalal PATEL, Vadodara (IN); Taslimarif SAIYED, Bengaluru (IN); Balachandran RAVINDRAN, Bhubaneswar (IN)

(73) Assignee: CENTRE FOR CELLULAR AND MOLECULAR PLATFORMS, Bengaluru, Karnataka (IN)

(21) Appl. No.: 18/280,434

(22) PCT Filed: Mar. 5, 2022
(86) PCT No.: PCT/IB2022/051960

§ 371 Date: Sep. 5, 2023

(30) Foreign Application Priority Data
Mar. 5, 2021 (IN) 202141009286

(51) Int. Cl. C07K 14/435; A61K 38/00; A61P 31/14; C07K 1/22; C07K 14/47; C12N 15/70

(52) U.S. Cl. CPC C07K 14/4354; A61P 31/14; C07K 1/22; C07K 14/47; C12N 15/70; A61K 38/00

(57) ABSTRACT

A recombinant heat shock protein (hsp70) or domains thereof is derived from filarial worm Setaria digitata or Homo sapiens so as to provide therapeutic and prophylactic anti-viral agents against respiratory viral infections. In that respect, a method of preparation of the recombinant heat shock protein (hsp70) is used. And the recombinant heat shock protein (hsp70) or domains thereof may be used for treatment and/or prophylaxis of one or more symptoms of respiratory viral infections in a subject.

### FIELD OF THE INVENTION

The present invention relates to a recombinant hsp70 and its domains as therapeutic and/or prophylactic anti-viral agent against respiratory viral infections. The invention provides recombinant hsp70 and its domains for immunomodulation in respiratory viral infections. The invention also relates to methods of preparation of said recombinant hsp70 and its domains and methods for therapeutically treating and/or preventing respiratory viral infections in a subject.

### BACKGROUND OF THE INVENTION

Respiratory viral infections like Influenza, Coronaviruses (SARS-CoV2) lead to various diseases in the respiratory organs. Infections with these viruses lead to detrimental effects on lungs and death in severe cases. These infections can cause viral pneumonia and acute respiratory distress syndrome (ARDS). The innate immune system has mechanisms to control these viruses upon infection and activation of these pathways can reduce the viral replication leading to improved outcomes. One of these pathways is dependent on toll like receptor signalling especially TLR2 and TLR4. Recently, prophylactic intranasal administration of a TLR2 agonist has been shown to reduce upper respiratory tract viral shedding in a SARS-CoV-2 in ferret model (Pamela C. Proud, 2020).

Hsp70 is a conserved molecule across organisms with varying roles. The intracellular hsp70 plays an important role as chaperone while its secretory version can activate immune receptors under stress. The host hsp70 protein is expressed in response to stress like a heat shock. Short-term heat shock has been shown to affect host-virus interaction in mice infected with highly pathogenic avian influenza virus H5N1 (Xue et al., 2016). This study has indicated that the innate immune response against the influenza virus is improved along with the increase in the intracellular levels of host hsp70.

The intracellular hsp70 has been shown to inhibit activity of Influenza-A virus ribonucleoprotein and to block its replication (Li G, 2011). In this study a recombinant version of human hsp70 fused to HIV-tat was used that enabled intracellular delivery of the recombinant protein. Protective role of host hsp70 has been shown in measles virus-infection model (Kim M Y, 2013).

The recombinant hsp70 derived from mammalian host or a filarial parasite as an immune-modulator to activate the TLR2/4 pathway has not been used before as an approach to control the viral replication.

U.S. Pat. No. 9,464,123B2 relates to peptides having activity of inhibiting infections of respiratory viruses. The peptides disclosed in the patent are synthesized by chemical or genetic engineering methods, and have functional domain capable of binding to surface glycoprotein of respiratory viruses and exhibit activity of inhibiting infections of respiratory viruses. The disclosed peptides are useful for blocking infections of respiratory viruses in target cells, for prevention/treatment of said infections, and for development of new prophylactic/therapeutic medicaments against respiratory viruses.

US2007/0087974 A1 discloses treatment or prevention of respiratory viral infections by administering an immuno-modulator compound of formula A to a subject.

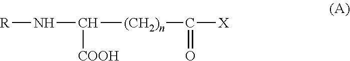

WO2014126508A1 discloses a pharmaceutical composition for treatment of local manifestations of herpes simplex virus infections and for prevention of influenza and acute respiratory viral infections, including green tea extract containing 70-90% epigallocatechin-Z-gallate (EGCG), colloidal silver and a gelling base.

WO2011127019A2 discloses method of treating, preventing, ameliorating, or delaying the onset of one or more symptoms associated with or resulting from a respiratory viral infection in a subject, comprising administering a compound, or a pharmaceutically acceptable salt, solvate, hydrate, or stereoisomer thereof, wherein the compound is a PDE4 modulator.

WO2004094991A2 discloses a method of treatment or prevention of a respiratory viral infection in a patient comprising administering to the patient an effective amount of an alpha thymosin peptide.

EP2187897A1 discloses pharmaceutical composition comprising effective dose of poly-gamma-glutamic acid for inhibiting viral infections or preventing viral diseases.

Hamidreza et al., 2014 discloses a recombinant protein from the extracellular domain of influenza A virus matrix protein 2 (M2e), fused with C-terminus of Mycobacterium tuberculosis Hsp70 (Hsp70c), to generate a vaccine candidate.

Li et al., 2011 discloses a recombinant human hsp70 proteins expressed as a fusion protein with Tat (a peptide that helps hsp70 to enter the cells).

Kim et al., 2013 discloses use of vesicular stomatitis virus (VSV) to establish relevance of hsp70-dependent antiviral immunity to fulminant cytopathic neuronal infections. In vitro, hsp70 that was constitutively expressed in mouse neuronal cells caused a modest increase in VSV replication. Infection induced an early extracellular release of hsp70 from viable cells, and the release was progressive, increasing with virus-induced apoptosis and cell lysis. The impact of this VSV-hsp70 interaction on neuro-virulence was established in weanling male hsp70 transgenic and non-transgenic mice. Constitutive expression of hsp70 in neurons of transgenic mice enhanced viral clearance from brain and reduced mortality, and it was correlated with enhanced expression of type I IFN mRNA. Non-transgenic mice were also protected against neuro-virulence and expressed increased type I IFN mRNA in brain when hsp70 was expressed by a recombinant VSV (rVSV-hsp70).

Advances in biotechnology have made available a large number of protein and peptide drugs for treatment of various diseases. Various drugs are not suitable for oral administration as they are subjected to degradation in gastrointestinal (GI) tract or considerably metabolized by first pass effect in liver. Intranasal drug delivery, out of many alternate routes, is much promising for administration of such drugs. Intra-nasal drug delivery route provides advantages of avoiding degradation of drug (as observed in GI tract), avoiding hepatic first pass metabolism, allowing rapid drug absorption thereby achieving quick onset of action, improved bioavailability of larger drug molecules by means of using absorption enhancer or other approach. Drugs that are not orally absorbed can be delivered to systemic circulation by nasal drug delivery (especially, for protein and peptide drugs), are convenient for patients (especially on long term therapy), have large nasal mucosal surface area for improved dose absorption, exhibit rapid drug absorption via highly-vascularized mucosa, are easy to administer, are non-invasive and lower dose/reduced side effects (Chhajed et al., 2011). Thus, nasal influenza vaccination is a good alternative to parenteral injection owing to enhancement of mucosal immune response and ease of vaccine administration.

US10159644B2 discloses influenza vaccine comprising a dry powder composition comprising a diketopiperazine and at least one of influenza virus-like particles, an antigenic protein, an antigenic peptide, fragments of said influenza virus-like particle, a subunit protein or multiple subunit proteins or fragments of said antigenic protein(s), fragments of said antigenic peptide, or combinations thereof.

WO2008037033A1 discloses a vaccine composition comprising a carbohydrate polymer comprising mannose and influenza virus (flu) antigen(s) in admixture.

US20190091324A1 discloses a method for preventing influenza in a human subject, comprising administering to the human subject an influenza vaccine composition comprising an inactivated whole influenza virion; and (ii) a gel base material comprising carboxy vinyl polymer.

CN1965844A discloses nasal medicine composition for prevention and treatment flu or influenza, the composition contains the atoxic pharmaceutically acceptable salt of the glycyrrhizic acid of 0.1-10% or glycyrrhizic acid as the active component.

Previous work in the field of art used various chemical compounds as antiviral agent against respiratory viral infections. Further, known candidates in the field of art which are suitable for nasal route of administration and demonstrate therapeutic and/or prophylactic effects against influenza virus comprise one or more live attenuated influenza viruses or components thereof. Such vaccines have restrictions of administration to selected individuals and need to be developed or improvised more frequently because influenza viruses are constantly mutating. Theses vaccines cannot be administered to pregnant females, humans with age 50 or older, having a weakened immune system due to disease or certain medical treatments, having a long-term health condition, such as diabetes, kidney disease, or heart or lung disease, including asthma and/or having a muscle or nerve condition that can cause problems with breathing or swallowing (such as epilepsy or cerebral palsy). The main disadvantages of these categories of vaccines are safety concerns in particular; live vaccines carry the risk of reversion to natural virulence via back-mutations of the attenuated organism and the possibility of causing symptoms similar to wild-virus infection in the recipient or in unprotected contacts (for instance, vaccine-associated paralytic poliomyelitis after oral poliovirus vaccine).

Thus, formulations against respiratory viral infections which are suitable for intranasal application and do not comprise viruses or any component thereof or harmful chemical entities are highly desirable. Therefore, there exists a need for formulations which can avoid restrictions and side effects of vaccine and drug candidates comprising virus or components thereof. Additionally, a need exists for formulations which provide advantages associated with nasal route of administration.

In the field of art, work related to hsp70 and anti-viral activity has been done on host intracellular protein. The antiviral activity of extracellular hsp70 delivered as recombinant proteins that can activate TLR2/4 pathways have not been explored.

Therefore, there is a need for therapeutic and/or prophylactic anti-viral agents against respiratory viral infections. The inventors of present invention studied protective (prophylactic and therapeutic) effects of recombinant hsp70 protein and its domains derived from human and filarial source. Setaria digitata is a filarial worm which infects cattle. Panda et al; 2012 showed that fraction made from total soluble worm lysate has affinity to WGA (wheat germ agglutinin) lectin (termed as AgW) and contains multiple glycoproteins which are able to bind to TLR4. The fraction was purified further and the underlying active component was identified in the Applicant's another patent application WO2021130729A1. The present invention prepared recombinant proteins and expressed the proteins in bacterial system and their efficacy was studied in mice influenza infection models and hamster SARS-CoV2 infection models.

Accordingly, the present invention provides recombinant hsp70 or its domains derived from filarial parasite or a mammal as antiviral agents. The present invention also provides recombinant hsp70 or its domains derived from filarial parasite or a mammal for therapeutic and/or prophylactic treatment of respiratory viral infections. The recombinant hsp70 or its domains of present invention are effective against respiratory viral infections and are suitable candidates for nasal route of administration. The invention further provides method of therapeutically and/or prophylactically treating a subject suffering from respiratory viral infections and methods of preparation of said recombinant hsp70 and its domains.

### REFERENCES

Chhajed S, Sangale S and Barhate S D (2011). Advantageous nasal drug delivery system: A review. DOI: http://dx.doi.org/10.13040/IJPSR.0975-8232.2 (6).1322-36.
Hamidreza A, Hassan N, Majid T (2014). Immunogenicity and protective efficacy of recombinant M2e.Hsp70c (Hsp70(359-610)) fusion protein against influenza virus infection in mice. Virol Sin. 2014 August; 29(4):218-27. doi: 10.1007/s12250-014-3428-8. Epub 2014 Aug. 18. DOI: 10.1007/s12250-014-3428-8.
Li G, Zhang J, Tong X, Liu W, Ye X (2011). Heat Shock Protein 70 Inhibits the Activity of Influenza A Virus Ribonucleoprotein and Blocks the Replication of Virus In Vitro and In Vivo. PLoS ONE 6(2): e16546. doi:10.1371/journal.pone.0016546.
Kim M Y, Ma Y, Zhang Y, Li J, Shu Y, Oglesbee M (2013). HSP70-Dependent Antiviral Immunity against Cytopathic Neuronal Infection by Vesicular Stomatitis Virus. Journal of Virology p. 10668-10678 October 2013 Volume 87 Number 19. DOI: 10.1128/JVI.00872-13.
Kim M Y, Shu Y, Carsillo T, Zhang J, Yu L, Peterson C, (2013). HSP70 and a novel axis of type I interferon-dependent antiviral immunity in the measles virus-infected brain. J Virol. 2013 January; 87(2):998-1009. doi: 10.1128/JVI.02710-12. Epub 2012 Nov. 7. PMID: 23135720; PMCID: PMC3554074.
Li G, Zhang J, Tong X, Liu W, Ye X (2011) Heat Shock Protein 70 Inhibits the Activity of Influenza A Virus Ribonucleoprotein and Blocks the Replication of Virus in vitro and in vivo. PLoS ONE 6(2): e16546. doi: 10.1371/journal.pone.0016546.
Pamela C. Proud, Daphne Tsitoura, Robert J. Watson et al (September 2020). Prophylactic intranasal administration of a TLR2 agonist reduces 1 upper respiratory tract viral shedding in a SARS-CoV-2 challenge 2 ferret model. BioRxiv preprint https://doi.org/10.1101/2020.09.25.309914).
Panda S K, Kumar S, Tupperwar N C, Vaidya T, George A, et al. Chitohexaose Activates Macrophages by Alternate Pathway through TLR4 and Blocks Endotoxemia. PLoS Pathog 8(5), 2012. 9. Berghe T V, Demon D, Bogaert P, Vandendriessche B, Goethals A, Depuydt B, et al. Simultaneous targeting of IL-1 and IL-18 is required for protection against inflammatory and septic shock. Am JRespir Crit Care Med. 189:282-91, 2014.
Xue J, FanX, YuJ, ZhangS, XiaoJ, HuY and Wang M (2016), Short-Term Heat Shock Affects Host-Virus Interaction in Mice Infected with Highly Pathogenic Avian Influenza Virus H5N1. Front.Microbiol.7:924. doi: 10.3389/fmicb.2016.00924.
Berghe T V, Demon D, Bogaert P, Vandendriessche B, Goethals A, Depuydt B, et al. Simultaneous targeting of IL-1 and IL-18 is required for protection against inflammatory and septic shock. Am J Respir Crit Care Med. (2014) 189:282-91. doi: 10.1164/rccm.201308-1535OC.
Davenport E E, Burnham K L, Radhakrishnan J, et al. Genomic landscape of the individual host response and outcomes in sepsis: a prospective cohort study. Lancet Respir Med. 2016; 4(4):259-271. doi:10.1016/S2213-2600(16)00046-1.
Eidt M V, Nunes F B, Pedrazza L, Caeran G, Pellegrin G, Melo D A, Possuelo L, Jost R T, Dias H B, Donadio M V, Oliveira J R. Biochemical and inflammatory aspects in patients with severe sepsis and septic shock: the predictive role of IL-18 in mortality. Clin. Chim. Acta. 2016; 453:100-106. doi: 10.1016/j.cca.2015.12.009.

### OBJECTS OF THE INVENTION

Accordingly, it is an object of the present invention to provide therapeutic and/or prophylactic anti-viral agents which act as immuno-modulators in respiratory viral infections.

It is also an object of the present invention to provide compositions and formulations comprising therapeutic and/or prophylactic anti-viral agents for immunomodulation in respiratory viral infections and to provide compositions and formulations demonstrating an improved efficacy for respiratory viral infections.

Another object of the present invention is to provide method of treatment of respiratory viral infections by therapeutic and/or prophylactic anti-viral agents.

Still another object of present invention is to provide suitable drug candidates against respiratory viral infections which can be administered via multiple routes.

It is also an object of present invention to provide suitable drug candidates against respiratory viral infections which can be administered by nasal route.

### SUMMARY OF THE INVENTION

The present invention provides recombinant hsp70 or its domains as therapeutic and prophylactic anti-viral agents against respiratory viral infections and methods of preparation of said recombinant hsp70 or its domains. The recombinant hsp70 and/or its domains are derived from mammalian host or a filarial parasite. The recombinant proteins and domains thereof act as immuno-modulators by activating TLR2/4 pathway. The present invention relates to antiviral activity of extracellular recombinant hsp70 proteins and domains thereof that can activate TLR2/4 pathways. The invention provides compositions and formulations comprising recombinant hsp70 proteins or domains thereof for treatment and/or prophylaxis of respiratory viral infections. The invention also provides method for therapeutically and/or prophylactically treating a subject suffering from respiratory viral infections comprising administering the compositions and formulations comprising recombinant hsp70 proteins or domains thereof.

In one of the embodiments, the present invention provides a recombinant heat shock protein (hsp70) or domains thereof derived from filarial worm Setaria digitata or Homo sapiens for treating or preventing a disease or disorder associated with respiratory viral infections wherein the recombinant hsp70 protein is of SEQ. ID NO. 1 or SEQ. ID NO. 2 or a variant thereof.

In another embodiment, the present invention provides that the domains of the hsp70 protein are C-terminal or N-terminal domains or a variant thereof.

In a further embodiment, the present invention provides that the variant is at least 80% identical to SEQ. ID NO. 1, SEQ. ID NO. 2, C-terminal or N-terminal domains, prior to or after alteration. In a yet another preferred embodiment, the present invention provides that the protein or domains thereof act as immuno-modulators by activating Toll like receptor TLR2/4 pathway.

In yet another embodiment the present invention provides a pharmaceutical formulation for treating and/or preventing a disease or disorder associated with respiratory viral infections, wherein the formulation comprises physiologically effective amount of recombinant hsp70 protein or domains thereof derived from Setaria digitata or Homo sapiens wherein the hsp70 protein is of SEQ. ID NO. 1 or SEQ. ID NO. 2 or a variant thereof.

In still another embodiment, the present invention provides that the domains of hsp70 protein in the formulation are C-terminal or N-terminal domains or a variant thereof. In another preferred embodiment, the present invention provides that the variant in the formulation is at least 80% identical to SEQ. ID NO. 1, SEQ. ID NO. 2, C-terminal or N-terminal domains, prior to or after alteration.

In another embodiment, the present invention provides that the physiologically effective amount of recombinant hsp70 protein or domains thereof in the formulation is 0.01 μg to 200 μg.

In yet another preferred embodiment, the present invention provides that the formulation immuno-modulates by activation of TLR4 and/or TLR2 receptors.

In still another preferred embodiment, the present invention provides that the formulation further comprises one or more suitable pharmaceutically acceptable additives, binders and excipients or a combination thereof.

In a further preferred embodiment, the present invention provides that the formulation is suitable for oral, parenteral, inhalation, dermal and intra-peritoneal mode of administration, preferably suitable for inhalation mode of administration.

In one of the embodiments the present invention also provides a method of preparing recombinant hsp70 protein or domains thereof comprising the following steps:
a) preparing a DNA construct comprising nucleotide sequence encoding one or more of recombinant hsp70 protein or domains thereof derived from filarial worm Setaria digitata or Homo sapiens;
b) constructing a bacterial expression vector comprising the DNA construct;
c) transforming a suitable prokaryotic host cell by said vector to obtain a transformed host cell;
d) culturing said transformed host cell in a suitable culture medium at suitable conditions for sufficient time selecting clones with high copy number of transformed gene from said transformed host cell and analyzing to expression of transformed gene of recombinant hsp70 protein or domains thereof;
e) subjecting the culture medium to suitable physical disruption technique followed by purifying expressed recombinant hsp70 protein or domains thereof.

In still embodiment, the present invention provides that the recombinant hsp70 protein in the method is derived from filarial worm Setaria digitata or Homo sapiens and is of SEQ. ID NO. 1 or SEQ. ID NO. 2 or a variant thereof.

In another embodiment, the present invention provides that the domains of hsp70 protein in the method are C-terminal or N-terminal domains or a variant thereof.

In still another embodiment, the present invention provides that the variant in the method is at least 80% identical to SEQ. ID NO. 1, SEQ. ID NO. 2, C-terminal or N-terminal domains, prior to or after alteration.

In yet another preferred embodiment, the present invention provides that the vector in the method is a pet28a or pet 22a bacterial expression vector.

In yet another preferred embodiment, the present invention provides that the purifying in step e) of the method is Ni-NTA based purification followed by removal of endotoxin.

In a further embodiment, the present invention provides a method of treatment, medicinal, curative, therapy and/or prophylaxis of disease or disorder associated with respiratory viral infections, the method comprising administering to a subject in need thereof, a physiologically effective amount of at least one of recombinant hsp70 protein or domains thereof derived from filarial worm Setaria digitata or Homo sapiens or a formulation or composition thereof.

In another embodiment, the present invention provides that the recombinant hsp70 protein or domains thereof in the method is capable of activating the TLR2/4 pathway. In a further preferred embodiment, the present invention provides that the physiologically effective amount of recombinant hsp70 protein or domains thereof in the method is 0.01 μg to 200 μg.

In one of the embodiments, the present invention further provides use of recombinant heat shock protein (hsp70) or domains thereof derived from filarial worm Setaria digitata or Homo sapiens of the present invention, or a formulation of the present invention, for the preparation of a medicament for preventing and/or treating respiratory viral infections in an individual.

In still another embodiment, the present invention provides use of recombinant heat shock protein (hsp70) or domains thereof derived from filarial worm Setaria digitata or Homo sapiens of the present invention, or a formulation of the present invention, for prevention and treatment of one or more symptoms of respiratory viral infections in an individual, comprising administering to the individual therapeutically effective amount of the formulation, wherein the formulation is capable of activating TLR2/4 pathway.

The foregoing general description and following detailed description are exemplary and explanatory and are intended to provide further explanation of the invention. Other embodiments, aspects, advantages, and features will be readily apparent to those skilled in the art from the following description of the invention.

### BRIEF DESCRIPTION OF THE DRAWINGS

FIG. 1 represents prophylactic effects of WFL (recombinant full-length protein or its domains derived from filarial worm Setaria digitata) and HFL (recombinant full-length protein or its domains derived from Homo sapiens) delivered intra-nasally 24 hour before Influenza infection. FIGS. 1A to 1C represents effect of indicated amounts of WFL and HFL on core temperature, body weight and survival of infected mice, respectively. FIG. 1D represents Influenza viral titer at the 15th days post infection. FIG. 1E demonstrates representative images from lungs of WFL and HFL treated mice.

FIG. 2 represents therapeutic effects of WFL and HFL delivered intra-nasally 24 hours after Influenza infection. FIGS. 2A to 2C represents effect of indicated amounts of WFL and HFL on core temperature, body weight and survival of infected mice, respectively.

FIG. 2D represents Influenza viral titre at the 15^{th} days post infection. FIG. 2E demonstrates representative images from lungs of WFL and HFL treated mice.

FIG. 3 represents therapeutic effects of WFL and HFL delivered intra-peritoneally 5 days after Influenza infection. FIGS. 3A to 3C represents effect of indicated amounts of WFL and HFL on core temperature, body weight and survival of infected mice, respectively. FIG. 3D represents Influenza viral titre at the 15^{th} days post infection. FIG. 3E demonstrates representative images from lungs of WFL and HFL treated mice.

FIG. 4 illustrates activity of WFL tested on golden hamster SARS-CoV2 infection model. FIG. 4A illustrates study plan used for testing the activity of WFL on SARS-CoV2 model. FIG. 4B illustrates viral copy number on day 4 in hamster model administered with WFL on day1 and day 2 post-infection.

FIG. 5 represents effect of different recombinant truncated versions of human and worm derived proteins on TLR reporter assays. FIG. 5A illustrates protein sizes and domains used for the reporter assay. HFL-Human Full Length, HNT-Human N-terminal, HCT-Human C-terminal, WFL-Worm Full Length, WNT-Worm N-terminal and WCT-Worm C-terminal. FIG. 5B illustrates effect of the different proteins on HEK-Blue TLR2 reporter cells. FIG. 5C illustrates effect of the different proteins on HEK-Blue TLR4 reporter cells.

FIG. 6 demonstrates size exclusion profile for AgW.

### DESCRIPTION OF THE INVENTION

The present invention investigates and demonstrates anti-viral activity of extracellular recombinant hsp70 proteins and/or domains thereof against respiratory viral infections. The recombinant hsp70 proteins or domains thereof were derived from filarial worm Setaria digitata and Homo sapiens. The hsp70 protein has three major functional domains; N-terminal ATPase domain (NBD domain) which binds ATP (Adenosine triphosphate) and hydrolyses it to ADP (Adenosine diphosphate), substrate binding domain composed of a β sheet subdomain and a helical subdomain and C-terminal domain. The recombinant proteins or domains thereof were cloned and expressed in bacterial expression vector. The expression vectors used for the pulse of invention were pet28a or pet 22a bacterial expression vector.

The expressed recombinant proteins or domains thereof were then purified. The invention used animal influenza models and SARS-CoV2 infection models for studying and comparing the therapeutic and prophylactic activity of recombinant proteins or domains thereof derived from filarial worm Setaria digitata and Homo sapiens. The animal models were administered with the formulations comprising recombinant hsp70 proteins or domains thereof through different routes of administration, including but not limited to, oral, parenteral, inhalation, dermal and intra-peritoneal mode of administration. In order to study dose-dependent prophylactic effect, the animal models were administered with varying doses of formulations/compositions comprising recombinant hsp70 proteins or domains thereof before influenza or SARS-CoV2 infection. In order to study dose-dependent therapeutic effect, the animal models were administered with varying dose of formulations/compositions comprising recombinant hsp70 proteins or domains thereof after influenza or SARS-CoV2 infection.

In order to study dose-dependent prophylactic and therapeutic effect of the formulations and compositions, the animal infection models were administered with formulations and compositions comprising recombinant hsp70 protein or domains thereof in 0.01 μg to 200 μg. The formulations and compositions of present invention can be in form of nasal spray which can be aqueous, hydroalcoholic, or nonaqueous-based solution, suspension, or emulsion system. The formulations of present invention can include a range of functional excipients, including but not limited to solvents and cosolvents; mucoadhesive agents; pH buffers; antioxidants; preservatives; osmolality and tonicity agents; penetration enhancers; suspending agents; and surfactants. The non-limiting examples of buffer used in the formulation are sodium phosphate, sodium citrate, citric acid.

The compositions of present invention can comprise excipients, including but not limited to an adjuvant; an osmotic pressure regulator, a pH regulator and a preservative. The osmotic pressure regulator includes at least one of sodium chloride, mannitol, potassium chloride, glycerine, sorbitol, glucose, and propylene glycol. The pH adjuster includes at least one of potassium dihydrogen phosphate, disodium hydrogen phosphate, citric acid, sodium citrate, sodium hydroxide, boric acid, and borax. The preservative comprises at least one of benzalkonium chloride, benzalkonium bromide, sorbic acid, methylparaben, ethylparaben, propylparaben, benzoic acid and benzyl alcohol.

The formulations or compositions can include variants of recombinant hsp70 protein or domains thereof. The variant is a functionally active variant and may be obtained by changing sequence of the hsp70 protein or domains thereof and is characterized by having a biological activity similar to that displayed by hsp70 protein of SEQ. ID NO.1 or SEQ. ID NO. 2 or domains of the hsp70 protein from which the variant is derived. The variant includes the ability of being capable of activating TLR2/4 pathway and/or to show protection against respiratory viral infections, wherein any variant may be tested in any of the tests described in the Examples.

The functionally active variant of hsp70 protein or domains thereof may be obtained by sequence alterations in sequence of hsp70 protein or domains thereof, wherein the peptide with the sequence alterations retains function of unaltered peptide. Such sequence alterations can include, but are not limited to, (conservative) substitutions, deletions, mutations and insertions. The variant can include functionally active fragment of the hsp70 protein or domains thereof, the functionally active fragment comprising at least 80% of the sequence of the hsp70 protein or domains thereof, preferably at least 85%, still more preferably at least 90%, even more preferably at least 95% and most preferably at least 97%, 98% or 99%.

The variant is derived from the hsp70 protein or domains thereof by at least one amino acid substitution and/or deletion, wherein the functionally active variant has a sequence identity to the hsp70 protein or domains thereof, or to the functionally active fragment as defined above of at least 80%, more preferably at least 85%, still more preferably at least 90%, even more preferably at least 95% and most preferably at least 97%, 98% or 99%.

The variant of the hsp70 protein or domains thereof is functionally active in the context of the present invention, if the activity of the variant amounts to at least 10%, preferably at least 25%, more preferably at least 50%, even more preferably at least 70%, still more preferably at least 80%, especially at least 90%, particularly at least 95%, most preferably at least 99% of the activity of the hsp70 protein or domains thereof without sequence alteration. The activity of the variant may be determined or measured as described in the Examples and then compared to that obtained for the hsp70 protein or domains thereof of the present invention.

The therapeutic efficacy of the recombinant hsp70 proteins or domains thereof at different time intervals after onset of influenza infection was investigated. The dose-dependent effect of administered recombinant proteins on core temperature, body weight and survival of infected mice was also investigated. The prophylactic and therapeutic effect of recombinant hsp70 proteins or domains thereof on respiratory viral infections was demonstrated by low viral load and reduced inflammation of lungs of animal models post influenza infection. The surprising prophylactic and therapeutic effects of WFL and HFL delivered intra-nasally in animal models demonstrate that they are suitable candidates for nasal drug delivery systems.

The invention demonstrates antiviral activity of extracellular recombinant hsp70 proteins by activating TLR2/4 pathways. Protective effects of a recombinant full version of the human hsp70/hspAIA (HFL) and filarial hsp70 (WFL) expressed in pet22a bacterial expression vector were tested in mice influenza infection models.

The recombinant hsp70 protein or domains thereof or a functionally active variant of hsp70 protein or domains thereof were administered in form of formulation or compositions wherein the ocnertaion vary from 0.01 μg to 200 μg. The recombinant proteins were used at 5 μg or 25 μg for intra-nasal application and at 25 μg or 50 μg for intra-peritoneal application. The prophylactic treatment of both WFL and HFL showed improvements over untreated mice and the effects of WFL were found to be better than HFL. The intranasal therapeutic treatment of both WFL and HFL also showed improvements over untreated mice. In addition, the therapeutic intervention with both HFL and WFL even after 5 days post infection showed improvements over untreated mice. Furthermore, the activity of 25 μg of WFL was found to be better than 25 μg HFL on survival whereas, the effect of both HFL and WFL were similar at 50 μg. The invention therefore demonstrates prophylactic and therapeutic activity of recombinant hsp70 in mice influenza model.

The protein sequences used are:

Heat Shock Protein 70 [Setaria digitata Hsp70]
The Heat shock protein hsp70 [Setaria digitata] has accession number as GenBank: >gi|4235279|gb|AAD13154.1 and has a sequence as shown below (SEQ. ID NO. 1)

MSKNAIGIDLGTTYSCVGVFMHGKVEIIANDQGNRTTPSYVAFTDTERLI
GDAAKNQVAMNPHNTVFDAKRLIGRKFDDGSVQSDMKHWPFKVMNAGGGK
PKVQVEYKGETKTFTPGEISSMVLVKMKETAEAFLGHAVKDAVITVPAYF
NDSQRQATKDSGAIAGLNVLRIINEPTAAAIAYGLDKKGHGERNVLIFDL
GGGTFDVSILTIEDGIFEVKSTAGDTHLGGEDFDNRMVNHFVAEFKRKHK
KDLASNPRALRRLRTACERAKRTLSSSSQASIEIDSLFEGIDFYTNITRA
RFEELCADLFRSTMDPVEKALRDAKMDKAQVHDIVLVGGSTRIPKVQKLL
SDFFSGKELNKSINPDEAVAYGAAVQAAILSGDKSEAVQDLLFVDVAPSL
GIETAGGVMTALIKRNTTIPTKTSQTFTTYSDNQPGVLIQVYEGERAMTK
DNNLLGKFELSGIPPAPRGVPQIEVTFDIDANGILNVSAQDKSTGKQNKI
TITNDKGRLSKDEIERMVQEAEKYKADDEAQKDRIAAKNALESYAFNMKQ
TIEDEKLRDKLSEEDKKKIQEKCDETVRWLDGNQTAEKDEFEHRQKELEA
VSNPIITKLYQSAGGMPGGMPGGMPGGAPGGGSGGSGPTIEEVD

HSPA1A Protein [Homo sapiens HSP70]

The Heat shock protein hspA1A [Homo sapiens] has accession number as >AAH18740.1 HSPA1A protein [Homo sapiens] and has a sequence as shown below (SEQ. ID NO. 2)

MAKAAAIGIDLGTTYSCVGVFQHGKVEIIANDQGNRTTPSYVAFTDTERL
IGDAAKNQVALNPQNTVFDAKRLIGRKFGDPVVQSDMKHWPFQVINDGDK
PKVQVSYKGETKAFYPEEISSMVLTKMKEIAEAYLGYPVTNAVITVPAYE
NDSQRQATKDAGVIAGLNVLRIINEPTAAAIAYGLDRTGKGERNVLIFDL
GGGTFDVSILTIDDGIFEVKATAGDTHLGGEDFDNRLVNHFVEEFKRKHK
KDISQNKRAVRRLRTACERAKRTLSSSTQASLEIDSLFEGIDFYTSITRA
RFEELCSDLFRSTLEPVEKALRDAKLDKAQIHDLVLVGGSTRIPKVQKLL
QDFFNGRDLNKSINPDEAVAYGAAVQAAILMGDKSENVQDLLLLDVAPLS
LGLETAGGVMTALIKRNSTIPTKQTQIFTTYSDNQPGVLIQVYEGERAMT
KDNNLLGRFELSGIPPAPRGVPQIEVTFDIDANGILNVTATDKSTGKANK
ITITNDKGRLSKEEIERMVQEAEKYKAEDEVQRERVSAKNALESYAFNMK
SAVEDEGLKGKISEADKKKVLDKCQEVISWLDANTLAEKDEFEHKRKELE
QVCNPIISGLYQGAGGPGPGGFGAQGPKGGSGSGPTIEEVD

The recombinant full-length (FL) filarial worm (Setaria digitata) and human (Homo sapiens) derived hsp70, named WFL and HFL respectively or domains thereof. Additionally, various truncated versions of recombinant WFL and HFL protein were prepared. The recombinant proteins were cloned in pet22a or pet28a bacterial expression vector. The cloned recombinant vector was transformed and expressed in a suitable host cell. The host cell used was preferably a prokaryotic cell and was cultured in suitable media under suitable conditions for its proliferation and expression.

The recombinant proteins were purified using the Ni-NTA based purification and the endotoxin was removed using the endotrap kit. In similar manner, recombinant domains of hsp70 protein were cloned in bacterial expression vector and expressed in suitable host cell followed by their purification.

### Example 1: Preparation of Recombinant Hsp70 Protein or Domains Thereof

### Isolation, Purification of Native Hsp Protein and Synthesis and Production of Recombinant Protein

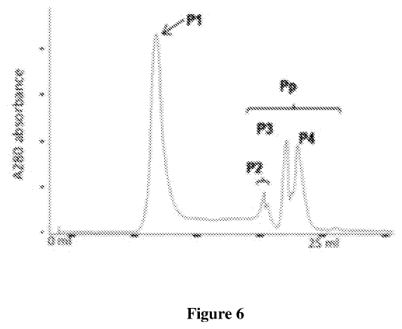

The Setaria digitata (Nematode) used for the purpose of this invention was peritoneal dwelling adult female filarial parasites obtained from cattle in a local abattoir, attached to the local zoological park at Nandankanan, Bhubaneswar after obtaining necessary approval from Zoo authorities. The AgW was fractionated on G75 size exclusion column where a major peak, P1 was seen apart from low peaks, P2, P3 and P4. As the quantity of other peaks was low they were pooled together as Pooled Peaks or Pp fraction (FIG. 6). The individual fractions were investigated for their ability to bind with and activate TLR4 and TLR2 immune receptors. There was significant seasonal variation in the AgW composition and the most potent fraction was named as P1 (peak 1).

The P1 fraction was found to be a pure glycoprotein and identified as Setaria digitata Heat Shock Protein 70 (SD-HSP70) by Mass Spectrometer base proteomic analysis. The recombinant version of SD-HSP70 was expressed in E. coli for scalability reasons. The E. coli cells used for the purpose of this invention were E. coli DH5alpha cells commercially procured from Thermo.

### Example 2: Cloning and Purification of Recombinant WFL and HFL or Domains Thereof

For Worm full-length (WFL) protein, Setaria Digitata worm was cut into smaller pieces and lysed through TRIzol (Invitrogen, USA) followed by RNA preparation. cDNA was made using RevertAid Reverse Transcriptase (Thermo Fisher Scientific, USA) and the PCR with filarial hsp70 specific primers with appropriate restriction sites [Forward Primer: GCAAGATCTATGTCAAAGAACGCAATC, (SEQ. ID NO. 3) and Reverse Primer: TGAGCGGCCGC CTAATCAACTTCTTCAAT (SEQ. ID NO. 4)] was performed using Phusion polymerase (Thermo Fisher Scientific, USA). The amplicon was cloned in pET28a vector that adds a His tag at the N-terminal. The human full length (HFL) gene sequence was subcloned from pcDNA5/FRT/TO HIS HSPA1A (a gift from Harm Kampinga, Addgene plasmid #19537; http://n2t.net/addgene: 19537; RRID: Addgene_19537) in the pET28a vector. The pET28a-WFL and pET28a-HFL clones were transformed in E. Coli BL21 strain; the cells were induced with IPTG overnight at 18° C. followed by sonication and purification using Ni-NTA resin (Cat. No #786-940, G-Biosciences, USA). The purity was confirmed by SDS-PAGE and the quantity was measured using NanoDrop 2000 (Thermo Fischer Scientific, USA). The pure protein was passed through EndoTrap® red endotoxin removal kit (LIONEX GmbH, Germany) followed by lyophilization.

Varying concentrations of the recombinant proteins or domains thereof were used for intra-nasal application and for intra-peritoneal application. The prophylactic and therapeutic effect of recombinant proteins or domains thereof was studied in mice influenza infection model and hamster SARS-CoV2 infection model.

### Example 3: Study of Prophylactic and Therapeutic Effects of Recombinant Hsp70 Protein or Domains Thereof

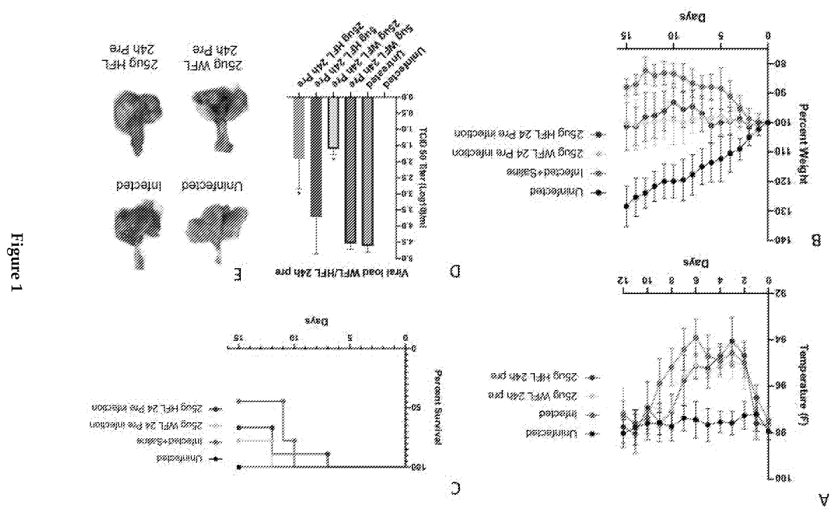

The prophylactic and therapeutic effects of recombinant hsp70 protein or domains thereof prepared in Examples 1 and 2 were studied in mice influenza model. For the prophylactic study, 5 μg or 25 μg of HFL and WFL were delivered through intranasal route before infection and the effects on temperature, body weight, survival and viral titers were observed over 15 days (FIGS. 1A-1D). The prophylactic treatment by both recombinant proteins WFL and HFL showed improvement over untreated mice and the effects of WFL were found to be better than HFL. The lung of the infected mice showed clear indication of inflammation, while the uninfected and both WFL and HFL treated mice lungs showed reduced inflammation (FIG. 1 E).

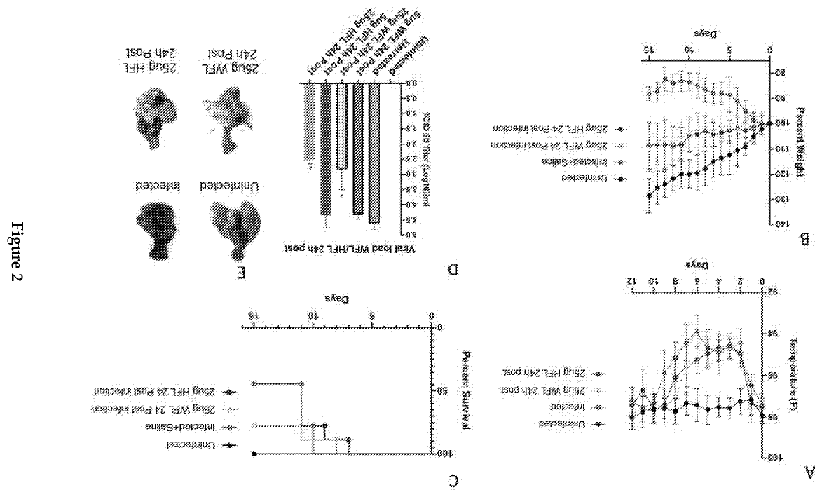

For the therapeutic study, 5 μg or 25 μg of HFL and WFL were delivered through intranasal route 24 hours after infection and the effects on temperature, body weight, survival and viral titers were observed over 15 days (FIGS. 2A-2D). The intranasal therapeutic treatment of both WFL and HFL showed improvements over untreated mice. The lungs of the infected mice showed clear indication of inflammation, while the uninfected and both WFL and HFL treated mice lungs showed reduced inflammation (FIG. 2 E).

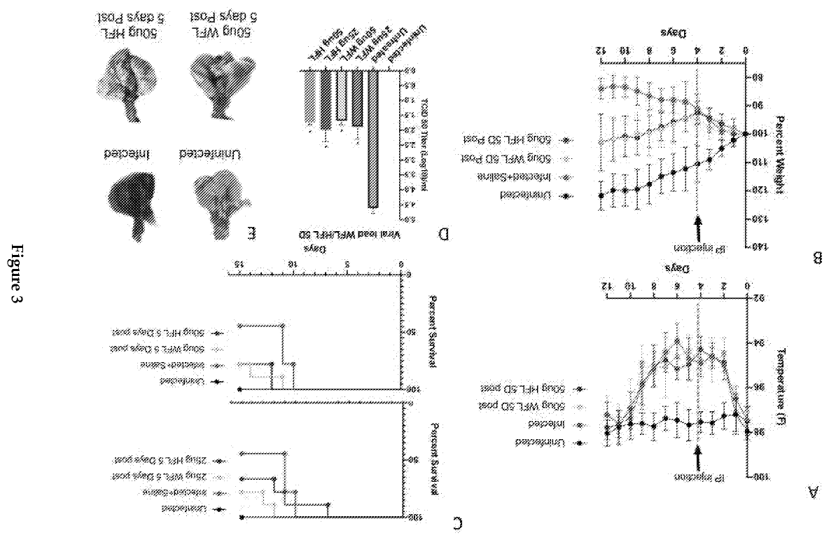

For therapeutic effects at late-stage infection, 25 μg or 50 μg of HFL and WFL were administered through intra-peritoneal route 5 days after infection and the effects on temperature, body weight, survival and viral titers were observed over 15 days (FIGS. 3A-3D). The therapeutic intervention with both HFL and WFL even after 5 days post infection showed improvements over untreated mice. The activity of 25 μg of WFL on survival was found to be better than 25 μg HFL. The effect of both HFL and WFL were similar at 50 μg. The lung of the infected mice showed clear indication of inflammation, while the uninfected and both WFL and HFL treated mice lungs showed reduced inflammation (FIG. 3 E).

The recombinant hsp70 protein or its domains of present invention can be used in formulations for therapeutically and prophylactically treating a subject suffering from respiratory viral infections. The recombinant proteins or domains thereof of present invention can be used more particularly as a nasal spray/drop for preventing infection and reducing severity of respiratory infections. The protein or domains thereof can also be used as a therapeutic agent later in the infection to reduce viral load and increasing the survival.

### Example 4: In Vitro Study of Efficacy of Recombinant Hsp70 Protein or Domains Thereof

### Cell Lines and Primary Cells

HEK-Blue TLR2 and HEK-Blue TLR4 reporter cells expressing SEAP (secreted embryonic alkaline phosphatase) enzyme under NF-kb and AP-1 promoter (Cat. no. #hkb-htlr2, hkb-htlr3 and hkb-htlr4) were purchased from InvivoGen (USA). HEK based cells were cultured in DMEM media. The cells were maintained at 37° C. incubator with 5% CO2. The culture media was supplemented with 10% Fetal Calf Serum (Gibco) along with standard antibiotics. HEK-Blue selection antibiotics (InvivoGen, Cat. no. #hb-sel, ant-zn and ant-bl) were added as per manufacturers' instructions.

### TLR Reporter Assays

2×10⁴ HEK-Blue reporter cells were seeded, in each well of a 96 well plate, in HEK-Blue detection media (InvivoGen, Cat. no. #hb-det3) along with05 nM and 10 nM of HFL, WFL or domains thereof. 10 ng/ml Pam3CSK4 (InvivoGen, Cat. no. #tlrl-pms.) and 10 ng/ml LPS-EK (InVivoGen, Cat. no. #tlrl-eklps), were used as a positive control for HEK-Blue TLR2 and TLR4 cells, respectively. The treated cells were incubated at about 37° C. for about 16 hours and absorbance (O.D.) was measured at 620 nm in either TECAN Infinite® 200 PRO or Thermo Scientific Varioskan LUX multimode reader.

### Results and Conclusions

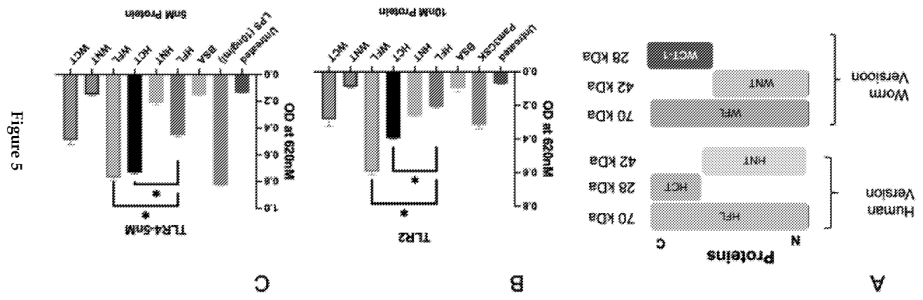

Effect of HFL-Human Full Length, HNT-Human N-terminal, HCT-Human C-terminal, WFL-Worm Full Length, WNT-Worm N-terminal and WCT-Worm C-terminal were studied on HEK-Blue TLR2 reporter cells at 10 nM dose (FIG. 5B). Effect of HFL-Human Full Length, HNT-Human N-terminal, HCT-Human C-terminal, WFL-Worm Full Length, WNT-Worm N-terminal and WCT-Worm C-terminal were studied on HEK-Blue TLR4 reporter cells at 5 nM dose (FIG. 5C).

The results of this study illustrate that C-terminal domain of human homologue HSP-70 (HCT) is responsible for TLR2/4 receptor activation. Removal of N-terminal region surprisingly increased activity as compared to full length human protein (HFL). Out of all proteins tested, WFL and the HCT demonstrated best activity. This was further tested in multiple biological replicates and results obtained therein indicated similar conclusions.

### Example 5: Study of Prophylactic and Therapeutic Effects of Recombinant Hsp70 Protein or Domains Thereof in Hamster SARS-CoV2 Infection Model

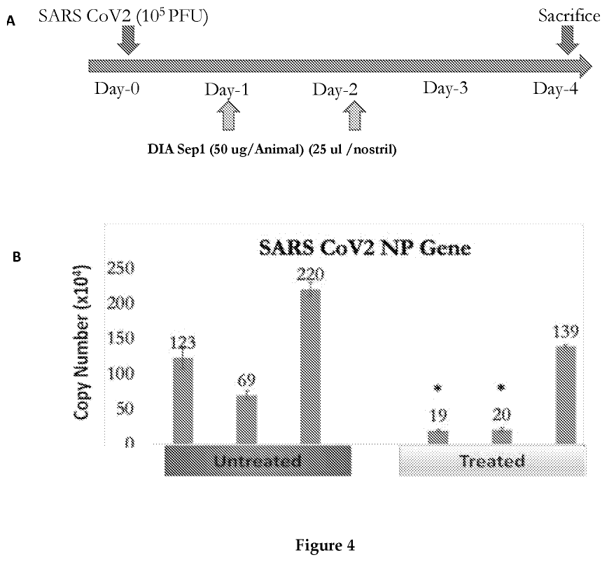

The prophylactic and therapeutic activity of recombinant hsp70 protein or domains thereof was further tested on hamster SARS-CoV2 model. The study plan is depicted in FIG. 4A. Golden hamsters were used for SARS-CoV2 infection as per the indicated copy number through the intranasal route and two doses of WFL were given through the intranasal route on day1 and day 2 post infection. The animals were sacrificed on day 4 and the viral titer was measured by RT-PCR in homogenised lung tissue.

### Results and Conclusions

The experimental data of FIG. 4B demonstrate that 2 out of 3 treated hamsters showed around 80-90% reduction in viral titers when compared to average titres from infected animals.

The WFL protein was tested in another pilot study in hamster model of SARS-CoV2 infection and the data in FIG. 4B illustrates that WFL reduces viral titer upon intranasal application post infection.

The present invention therefore provides therapeutic and prophylactic anti-viral agents which act as immuno-modulators in respiratory viral infections. The results of present invention illustrate that recombinant hsp70 proteins and domains thereof show unexpected and surprising prophylactic as well as therapeutic activity against respiratory viral infections. The unexpected and surprising prophylactic as well as therapeutic activity of recombinant hsp70 proteins and domains thereof against influenza and SARS-CoV2 establishes them as suitable drug candidates against respiratory viral infections. The present invention provides recombinant hsp70 proteins and domains thereof for use in method of treatment, prophylaxis, therapy of respiratory viral infections.

The recombinant hsp70 proteins and domains thereof can be useful as compositions and formulations for immunomodulation in respiratory viral infections. The compositions and formulations of recombinant hsp70 proteins and domains thereof can be administered via multiple routes and preferably via nasal route.

The present invention has been described with reference to the exemplary embodiments and the drawings, but the present invention is not limited thereto. The invention may be embodied in other specific forms without departing from the spirit or essential characteristics thereof. The foregoing embodiments are therefore to be considered in all respects illustrative rather than limiting the invention described herein. It will be obvious to those skilled in the art to make various changes, modifications and alterations to the invention described herein. To the extent that these various changes, modifications and alteration do not depart from the scope of the present invention, they are intended to be encompassed therein. In addition, where this application has listed the steps of a method or procedure in a specific order, it may be possible, or even expedient in certain circumstances, to change the order in which some steps are performed, and it is intended that the particular steps of the method or procedure claims set forth herein below not be construed as being order-specific unless such order specificity is expressly stated in the claim. Scope of the invention is thus indicated by the appended claims rather than by the foregoing description, and all changes that come within the meaning and range of equivalency of the claims are intended to be embraced therein.

1. A recombinant heat shock protein (hsp70) or domains thereof derived from filarial worm Setaria digitata or Homo sapiens for treating or preventing a disease or disorder associated with respiratory viral infections wherein the recombinant hsp70 protein is of SEQ. ID NO. 1 or SEQ. ID NO. 2 or a variant thereof.

2. The recombinant hsp70 protein or domains thereof as claimed in claim 1, wherein the domains of the hsp70 protein are C-terminal or N-terminal domains or a variant thereof.

3. The recombinant hsp70 protein or domains thereof as claimed in claim 1, wherein the variant is at least 80% identical to SEQ. ID NO. 1, SEQ. ID NO. 2, C-terminal or N-terminal domains, prior to or after alteration.

4. The recombinant hsp70 protein or domains thereof as claimed in claim 1, wherein the protein or domains thereof act as immuno-modulators by activating Toll like receptor TLR2/4 pathway.

5. A pharmaceutical formulation for treating and/or preventing a disease or disorder associated with respiratory viral infections, wherein the formulation comprises physiologically effective amount of the recombinant hsp70 protein or domains as claimed in claim 1.

6. The pharmaceutical formulation as claimed in claim 5, wherein the domains of hsp70 protein are C-terminal or N-terminal domains or a variant thereof.

7. The pharmaceutical formulation as claimed in claim 5, wherein the variant is at least 80% identical to SEQ. ID NO. 1, SEQ. ID NO. 2, C-terminal or N-terminal domains.

8. The pharmaceutical formulation as claimed in claim 5, wherein the physiologically effective amount of recombinant hsp70 protein or domains thereof is 0.01 μg to 200 μg.

9. The pharmaceutical formulation as claimed in claim 5, wherein the formulation immuno-modulates by activation of TLR4 and/or TLR2 receptors.

10. The pharmaceutical formulation as claimed in claim 5, further comprising of one or more suitable pharmaceutically acceptable additives, binders and excipients or a combination thereof.

11. The pharmaceutical formulation as claimed in claim 5, wherein the formulation is suitable for oral, parenteral, inhalation, dermal and intra-peritoneal mode of administration.

12. A method of preparing recombinant hsp70 protein or domains thereof comprising the following steps:
preparing a DNA construct comprising nucleotide sequence encoding one or more of recombinant hsp70 protein or domains thereof derived from filarial worm Setaria digitata or Homo sapiens;
constructing a bacterial expression vector comprising the DNA construct;
transforming a suitable prokaryotic host cell by said vector to obtain a transformed hostcell;
culturing said transformed host cell in a culture medium to express transformed gene of recombinant hsp70 protein or domains thereof;
subjecting the culture medium to suitable physical disruption technique followed by purifying expressed recombinant hsp70 protein or domains thereof.

13. The method as claimed in claim 12, wherein the recombinant hsp70 protein is derived from filarial worm Setaria digitata or Homo sapiens and is of SEQ. ID NO. 1 or SEQ. IDNO. 2 or a variant thereof.

14. The method as claimed in claim 12, wherein the domains of hsp70 protein are C-terminal or N-terminal domains or a variant thereof.

15. The method as claimed in claim 13, wherein the variant is at least 80% identical to SEQ. ID NO. 1, SEQ. ID NO. 2, C-terminal or N-terminal domains, prior to or after alteration.

16. The method as claimed in claim 12, wherein the vector is a pet28a or pet22a bacterial expression vector.

17. The method as claimed in claim 12, wherein the purifying in step e) is Ni-NTA based purification followed by removal of endotoxin.

18. A method of treatment, medicinal, curative, therapy and/or prophylaxis of disease or disorder associated with respiratory viral infections, the method comprising administering to a subject in need thereof, a physiologically effective amount of at least one of recombinant hsp70 protein or domains thereof derived from filarial worm Setaria digitata or Homo sapiens or a formulation or composition thereof.

19. The method as claimed in claim 18, wherein the recombinant hsp70 protein or domains thereof is capable of activating the TLR2/4 pathway.

20. The method as claimed in claim 18, wherein the physiologically effective amount of recombinant hsp70 protein or domains thereof is 0.01 μg to 200 μg.

21. (canceled)

22. (canceled)


SEQUENCE LISTING (4 sequences)

SEQ ID NO: 1 - protein, 645 aa, Artificial Sequence
  Recombinant hsp70 protein sequence obtained from Setaria digitata
MSKNAIGIDLGTTYSCVGVFMHGKVEIIANDQGNRTTPSYVAFTDTERLIGDAAKNQVAMNPHNTVFDAKRLIGRKFDDGSVQSDMKHWPFKVMNAGGGKPKVQVEYKGETKTFTPGEISSMVLVKMKETAEAFLGHAVKDAVITVPAYFNDSQRQATKDSGAIAGLNVLRIINEPTAAAIAYGLDKKGHGERNVLIFDLGGGTFDVSILTIEDGIFEVKSTAGDTHLGGEDFDNRMVNHFVAEFKRKHKKDLASNPRALRRLRTACERAKRTLSSSSQASIEIDSLFEGIDFYTNITRARFEELCADLFRSTMDPVEKALRDAKMDKAQVHDIVLVGGSTRIPKVQKLLSDFFSGKELNKSINPDEAVAYGAAVQAAILSGDKSEAVQDLLFVDVAPLSLGIETAGGVMTALIKRNTTIPTKTSQTFTTYSDNQPGVLIQVYEGERAMTKDNNLLGKFELSGIPPAPRGVPQIEVTFDIDANGILNVSAQDKSTGKQNKITITNDKGRLSKDEIERMVQEAEKYKADDEAQKDRIAAKNALESYAFNMKQTIEDEKLRDKLSEEDKKKIQEKCDETVRWLDGNQTAEKDEFEHRQKELEAVSNPIITKLYQSAGGMPGGMPGGMPGGAPGGGSGGSGPTIEEVD

SEQ ID NO: 2 - protein, 641 aa, Artificial Sequence
  Recombinant hspAIA protein sequence obtained from Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 3 - DNA, 27 bp, Artificial Sequence
  Forward Primer
gcaagatctatgtcaaagaacgcaatc

SEQ ID NO: 4 - DNA, 29 bp, Artificial Sequence
  Reverse Primer
tgagcggccgcctaatcaacttcttcaat